SEQUENCE LISTING (3 sequences)

SEQ ID NO: 1 - DNA, 21 bp, Influenza A virus
tgctttactgtaatgaccgat

SEQ ID NO: 2 - DNA, 21 bp, Influenza A virus
gattctggttgaaagacaccc

SEQ ID NO: 3 - DNA, 22 bp, Influenza A virus
tagaatcaggataacaggagca

(19) United States
(12) Patent Application Publication
(10) Pub. No.: US 2012/0308993 A1
(43) Pub. Date: Dec. 6, 2012

(54) RESISTANT INFLUENZA A VIRUS DETECTION METHOD AND KIT THEREFOR

(75) Inventors: Masafumi Inoue, Singapore (SG); Timothy Barkham, Singapore (SG)

(73) Assignees: TAN TOCK SENG HOSPITAL, Singapore (SG); AGENCY FOR SCIENCE, TECHNOLOGY AND RESEARCH, Singapore (SG)

(21) Appl. No.: 13/505,900

(22) PCT Filed: Nov. 8, 2010
(86) PCT No.: PCT/SG2010/000425

§ 371 Date: Jul. 11, 2012

(30) Foreign Application Priority Data
Nov. 5, 2009 (SG) 200907424-6

(51) Int. Cl. C12Q 1/70; C12P 19/34; C07H 21/04

(52) U.S. Cl. 435/5; 536/24.3; 536/24.33; 536/23.72; 435/91.2

(57) ABSTRACT

There is provided a method of detecting the presence of oseltamivir-resistant Influenza A virus in a sample, the method comprising detecting the presence of an amplicon, a fragment and/or a derivative thereof, wherein the amplicon, fragment and/or derivative thereof (a) is amplified by a primer (SEQ ID NO:1), a fragment, or derivative therefore and/or a primer (SEQ ID NO:2), a fragment, or derivative thereof. There are also provided primer(s) and/or probe as well as kit for the detection of the presence of Influenza A in a sample and/or in a subject.

### FIELD OF THE INVENTION

The present invention relates to primer(s), probes as well as method(s) and kit(s) using such primer(s) and/or probes for the detection of the presence of Influenza A virus. In particular, the invention relates to the detection of anti-viral drug resistant influenza A virus, subtype H1N1.

### BACKGROUND TO THE INVENTION

Influenza A is an infectious disease of animals caused by type A strains of the influenza virus that normally infect birds, and less commonly, pigs. There are many subtypes of the influenza A virus. These subtypes are based on the haemagglutinin (HA) segment 4, which has 14 varieties and neuraminidase (NA) segment 6, which has 9 varieties. It is these two segments of the virus that cause virulence.

For example, influenza A subtype, H1N1, is known to mutate quickly, and has been shown to be highly pathogenic and can cause severe diseases in man. H1N1 has been viewed as a model for rapid genetic evolution and pandemic change. Generally, evolution is explained to be the selection of frequent copy errors generated by a polymerase complex that lacks a copy function. These errors are then selected for an evolutionary advantage, such as evasion of the immune response of the host, which allows the influenza to expand and fix the selected mutation. Also, influenza viruses employ recombination for rapid evolution via homologous recombination. Therefore, mutation in influenza occurs frequently and these variants may include properties such as anti-viral drug resistance, ability to escape from immune recognition and the like. Further, a study on 64 children in the United Kingdom demonstrated a higher rate of drug resistance in H1N1 than H3N2 and influenza B.

One of the more commonly used anti-viral drugs in the past was oseltamivir. It was used as monotherapy for seasonal H1N1. However, the effectiveness of oseltamivir dropped with the emergence of H1N1 with resistance to oseltamivir. In each reported instance, H1N1 was isolated with the neuraminidase polymorphism, H274Y. This change was expected, because it is located in the active site of the enzyme, and oseltamivir binding requires a conformational change in the active site and H274Y inhibits this change leading to a strain with oseltamivir resistance.

Seasonal H1N1 viruses with the H275Y mutation became widespread during the 2007-2008 northern hemisphere influenza seasons. In 2007-2008, the H275Y mutation was detected in 64% of influenza A (H1N1) isolates collected from Oceania, South East Asia, and South Africa (Hurt, 2009). Patients infected with the oseltamivir resistant virus were more likely to develop pneumonia or sinusitis than those infected with the wild-type virus. It was widely postulated that the emergence of resistance in seasonal H1N1 was due to the selective pressure of drug therapy and that resistant virus was likely to be less fit for disease transmission. However, resistant seasonal H1N1 rapidly replaced susceptible wild type seasonal H1N1 in the face of a relatively low consumption of oseltamivir in Europe between 2002 and 2007 (Kramarz P et al., 2009).

More recently, in the 2009 flu pandemic, the new strain of virus, Influenza A (H1N1) 2009, isolated was found to be made up of genetic elements from four different flu viruses—North American swine influenza, North American avian influenza, human influenza, and swine influenza virus typically found in Asia and Europe. This new strain appears to be a result of reassortment of human influenza and swine influenza viruses, in all four different strains of subtype H1N1.

The principle mutation (H275Y) associated with oseltamivir resistance in vitro has also been recently reported in at least 39 cases of H1N1 (WHO Pandemic (H1N1) 2009—update 71). The clinical importance of this mutation in this virus is assumed from in vitro data. The worldwide prevalence of this mutation in seasonal H1N1 rapidly increased from being negligible in 2007 to up to 95% as of March 2009 (http://www.who.int/csr/disease/influenza/h1n1_table/en/index.html).

Influenza A (H1N1) 2009 was first detected in Singapore in May 2009. In an attempt to slow down the spread of the virus affected patients were kept in isolation until the virus could no longer be detected by PCR, which was taken as a surrogate marker of infectivity. Surveillance for the H275Y mutation was systematically performed on samples from new cases.

The spontaneous emergence of the oseltamivir-resistant seasonal H1N1 was not surprising but its dissemination, prevalence, and persistence were unexpected. The real question was whether oseltamivir would fail when used as monotherapy in a life threatening case of influenza with the H275Y mutation. We assumed it would fail for seasonal H1N1 and expect the same for Influenza A (H1N1) 2009. It is therefore of great interest to consider whether resistant mutants of Influenza A (H1N1) 2009 will mirror the global expansion of seasonal H1N1 in 2007-2009 and expand to become the dominant Influenza virus, replacing both the current wild type ‘susceptible’ H1N1 virus and the normally predominant H3N2 viruses.

### SUMMARY OF THE INVENTION

With the rapid emergence of new strains of Influenza A viruses with mutations in the genome leading to properties that allow better survival of the virus in the body, early detection and treatment is essential to eradicate the virus. Molecular biology techniques like PCR and RT-PCR may be used for the detection of the virus. However, since the Influenza A viruses are prone to mutations, the sensitivity of the primers used is fundamental. Also, as the emergence of resistant virus is of public health importance, specific and/or sensitive detection method(s) for anti-viral resistant influenza A virus, is essential.

The severity of disease due to Influenza A (H1N1) 2009 is in general similar to that of seasonal H1N1, with only a small proportion of infections producing severe disease. However, the concern is that there may be huge numbers of influenza due to the immunologically naive younger global population and that with this large denominator the small proportion of severe cases may actually represent a considerable cohort. In these cases anti-viral therapy and the development of resistance may be quite important. If resistant mutants become prevalent, as they did with seasonal H1N1, then resistance testing will be required but laboratories will not be able to support these requests as the necessary technology is relatively unavailable; empirical monotherapy with oseltamivir would became less comfortable and practice may quickly assume either the use of zanamivir as monotherapy or dual cover with oseltamivir. Accordingly, specific and/or sensitive detection method(s) for anti-viral resistant Influenza A (H1N1) 2009, is essential.

The present invention provides highly sensitive and specific oligonucleotides, fragment(s) and/or derivative(s) thereof useful in a method of detecting Influenza A virus in patient specimens. The oligonucleotides of the present invention may be capable of obtaining high yield of amplicon by single gene amplification and provide rapid and cost-effective diagnostic and prognostic reagents for determining infection by influenza A viruses and/or disease conditions associated therewith. In particular, the oligonucleotides of the present invention may be used to detect anti-viral drug resistant influenza A viruses. The anti-viral drug that the influenza A virus strains may be resistant to may be a neuraminidase inhibitor such as for example, oseltamivir, zanamivir, peramivir, or analogs thereof. In particular, the influenza A virus may be resistant to oseltamivir. More in particular, the anti-viral drug resistant influenza A virus may be of subtype H1N1. These primers may provide an informative influenza assay that can be performed in the field, i.e., at the point of care (“POC”).

According to a first aspect, the present invention provides at least one isolated oligonucleotide comprising, consisting essentially of, or consisting of at least one nucleotide sequence selected from the group consisting of: SEQ ID NO:1 to SEQ ID NO:3, fragment(s), derivative(s), mutation(s), and complementary sequence(s) thereof. The oligonucleotide may be capable of binding to and/or being amplified from influenza A virus. The influenza A virus may be an anti-viral drug resistant strain. In particular, the anti-viral drug may be oseltamivir. The anti-viral drug resistant strain may be of subtype H1N1.

According to another aspect, the present invention provides at least one pair of oligonucleotides comprising at least one forward primer and at least one reverse primer, wherein the forward primer comprises, consists essentially of or consists of SEQ ID NO:1, fragment(s), derivative(s), mutation(s), and complementary sequence(s) thereof and the reverse primer comprises, consists essentially of, or consists of SEQ ID NO:2, fragment(s), derivative(s), mutation(s), and complementary sequence(s) thereof.

According to one aspect, the present invention provides a set of oligonucleotides comprising a pair of oligonucleotides according to any aspect of the present invention and at least one probe.

According to yet another aspect, the present invention provides an amplicon amplified from Influenza A using at least one forward primer comprising, consisting of or consisting essentially of the nucleotide sequence of SEQ ID NO:1, fragment(s), derivative(s), mutation(s), and complementary sequence(s) thereof and at least one reverse primer comprising, consisting of or consisting essentially of the nucleotide sequence of SEQ ID NO:2, fragment(s), derivative(s), mutation(s), and complementary sequence(s) thereof.

According to one aspect, the present invention provides at least one method of detecting the presence of influenza A virus in a biological sample, the method comprising the steps of:
(a) providing at least one biological sample;
(b) contacting at least one oligonucleotide, pair of oligonucleotides or set of oligonucleotides according to any aspect of the present invention, with at least one nucleic acid in the biological sample, and/or with at least one nucleic acid extracted, purified and/or amplified from the biological sample; and
(c) detecting any binding resulting from the contacting in step (b) whereby the influenza A virus is present when binding is detected.

The influenza A virus may be an anti-viral drug resistant strain. In particular, the anti-viral drug may be oseltamivir. The anti-viral drug resistant strain may be of subtype H1N1.

According to one aspect, the present invention provides at least one method of amplifying anti-viral drug resistant influenza A virus nucleic acid, wherein said method comprises carrying out a polymerase chain reaction using at least one forward primer according to any aspect of the present invention and a reverse primer according to any aspect of the present invention. The anti-viral drug resistant strain may be of subtype H1N1.

According to another aspect, the present invention provides at least one kit for the detection of anti-viral drug resistant influenza A virus, the kit comprising at least one oligonucleotide or a pair of oligonucleotides according to any aspect of the present invention and/or a sequencing primer according to any aspect of the present invention. The anti-viral drug resistant strain may be of subtype H1N1. All primers and probes may be mixed to construct multiplex one-step fluorescence probe-based real-time PCR in one-tube with high specificity and high sensitivity.

According to a particular aspect, there are provided highly sensitive and specific primers, fragments and/or derivatives thereof useful in a method of PCR capable of detecting oseltamivir resistant influenza A virus DNA in patient specimens. This test may be used to examine the specimens from patients with influenza A and in particular oseltamivir resistant influenza A of subtype H1N1. The primers may be sensitive and specific. Further, at least one IC molecule may be included in each reaction to monitor the PCR performance.

As will be apparent from the following description, preferred embodiments of the present invention allow an optimal use of the oligonucleotides to take advantage of the specificity and selectivity of these primers. This and other related advantages will be apparent to skilled persons from the description below.

### BRIEF DESCRIPTION OF THE FIGURES

FIG. 1 shows the results of a clinical examination of a patient, 28-year-old female American naval officer. The results show the hourly trend of temperature, clinical symptoms, PCR test, positivity, and oseltamivir treatment whereby “1” denotes oseltamivir served or clinical symptom reported or positive PCR test result and “0” denotes absence.

FIG. 2 shows the pyrosequencing sequences (Pyrogram traces of H275Y analyses). The sequence read is GT(G/A)ATA in each plot. The shaded area indicates the mutation site. The G peak in the shaded box varies between 0 and 1 (normalized units), whereas the A peak after the G varies between 1 and 2, since the subsequent nucleotide in the sequence is A.

(a) Readings taken on 27th May, Directly on sample; 100% wild G;

(b) Readings taken on 27th May, viral isolate; 100% wild G;

(c) Readings taken on 29th May, Directly on sample; 100% wild G;

(d) Readings taken on 30th May, Directly on 38 hr sample; Wild G is 76% and mutant A is 24%;

(e) Readings taken on 30th May, Directly on 45 hr sample; Wild G is 46% and mutant A is 52%; and

(f) Readings taken on 30th May, Cultured sample; 100% mutant A.

### DETAILED DESCRIPTION OF THE PREFERRED EMBODIMENTS

Bibliographic references mentioned in the present specification are for convenience listed in the form of a list of references and added at the end of the examples. The whole content of such bibliographic references is herein incorporated by reference.

### DEFINITIONS

The term “biological sample” is herein defined as a sample of any tissue and/or fluid from at least one animal and/or plant. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc. Environmental samples include environmental material such as surface matter, soil, water, air and industrial samples, as well as samples obtained from food and dairy processing instruments, apparatus, equipment, utensils, disposable and non-disposable items. These examples are not to be construed as limiting the sample types applicable to the methods disclosed herein. In particular, a biological sample may be of any tissue and/or fluid from at least a human being.

The term “complementary” is used herein in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” The degree of complementarity between nucleic acid strands has significant effects on the efficiency and strength of hybridization between nucleic acid strands. This is of particular importance in amplification reactions, as well as detection methods that depend upon binding between nucleic acids. In particular, the “complementary sequence” refers to an oligonucleotide which, when aligned with the nucleic acid sequence such that the 5′ end of one sequence is paired with the 3′ end of the other, is in “anti-parallel association.” Certain bases not commonly found in natural nucleic acids may be included in the nucleic acids disclosed herein and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. Where a first oligonucleotide is complementary to a region of a target nucleic acid and a second oligonucleotide has complementary to the same region (or a portion of this region) a “region of overlap” exists along the target nucleic acid. The degree of overlap may vary depending upon the extent of the complementarity.

The term “comprising” is herein defined as “including principally, but not necessarily solely”. Furthermore, the term “comprising” will be automatically read by the person skilled in the art as including “consisting of”. The variations of the word “comprising”, such as “comprise” and “comprises”, have correspondingly varied meanings.

The term “derivative,” is herein defined as the chemical modification of the oligonucleotides of the present invention, or of a polynucleotide sequence complementary to the oligonucleotides. Chemical modifications of a polynucleotide sequence can include, for example, replacement of hydrogen by an alkyl, acyl, or amino group.

The term “fragment” is herein defined as an incomplete or isolated portion of the full sequence of an oligonucleotide which comprises the active/binding site(s) that confers the sequence with the characteristics and function of the oligonucleotide. In particular, it may be shorter by at least one nucleotide or amino acid. More in particular, the fragment comprises the binding site(s) that enable the oligonucleotide to bind to influenza virus. A fragment of the oligonucleotides of the present invention may be about 20 nucleotides in length. In particular, the length of the fragment may be at least about 10 nucleotides in length. For example, the fragment of the forward primer may comprise at least 10, 12, 15, 18 or 19 consecutive nucleotides of SEQ ID NO:1, and/or the reverse primer may comprise at least 10, 12, 15, 18, 19, 20, 22, or 24 consecutive nucleotides of SEQ ID NO:2, More in particular, the fragment of the primer may be at least 15 nucleotides in length.

The term ‘H275Y’ is herein defined as the mutation in the gene in the Influenza A virus which would result in the original amino acid at position 275 along the neuroaminidase protein changing from the expected wild type ‘H’ (Histidine) to ‘Y’ (Tyrosine). The actual change in the Influenza gene is a single point mutation from a Cytosine base to a Thymine base.

The term “internal control (IC) molecule” is herein defined as the in vitro transcribed oligonucleotide molecule which is co-amplified by the same primer set for influenza virus used in the method of the present invention. In particular, the IC may be mixed in the reaction mixture to monitor the performance of PCR to avoid false negative results. The probe to detect this IC molecule may be specific to the interior part of this molecule. This interior part may be artificially designed and may not occur in nature.

The term “influenza virus” as used in the context of the invention includes all subtypes of influenza viruses that fall under the categories of “avian influenza viruses” and “human influenza viruses”. The influenza viruses may be influenza A, B or C viruses. In particular, the influenza A virus may include but are not limited to H1N1, H3N2, H5N1, H5N2, H5N8, H5N9, H7N2, H7N3, H7N4, H7N7, H9N2 and the like.

The term “mutation” is herein defined as a change in the nucleic acid sequence of a length of nucleotides. A person skilled in the art will appreciate that small mutations, particularly point mutations of substitution, deletion and/or insertion has little impact on the stretch of nucleotides, particularly when the nucleic acids are used as probes. Accordingly, the oligonucleotide(s) according to the present invention encompasses mutation(s) of substitution(s), deletion(s) and/or insertion(s) of at least one nucleotide. Further, the oligonucleotide(s) and derivative(s) thereof according to the present invention may also function, as probe(s) and hence, any oligonucleotide(s) referred to herein also encompasses their mutations and derivatives. For example, if mutations occur at a few base positions at any primer hybridization site of the target gene, particularly to the 5′-terminal, the sequence of primers may not affect the sensitivity and the specificity of the primers.

The term “nucleic acid in the biological sample” refers to any sample that contains nucleic acids (RNA or DNA). In particular, sources of nucleic acids are biological samples including, but not limited to blood, saliva, cerebral spinal fluid, pleural fluid, milk, lymph, sputum and semen.

The oligonucleotides according to the present invention may be useful as primers and/or probes and may be used in methods for specifically detecting anti-viral drug resistant influenza A in a sample containing either one or both strains of influenza and/or other unrelated viruses/microscopic organisms. These nucleotide sequences of the primers and probes of the present invention are designed to hybridize specifically to regions of the resistant influenza A genomes that are unique to the genome of each strain, but which are also conserved across many viruses within each strain.

The primers according to any aspect of the present invention may be used to distinguish the genotype of drug resistant influenza A from non-drug resistant influenza A. In particular, the primers according to the present invention may be used to distinguish the genotype of the different subtypes of drug resistant influenza A virus. Even more in particular, the primers may be used to distinguish influenza A with swine-origin H1N1 subtyping confirmation, seasonal H1N1, seasonal H3N2 and the like.

The drug resistant strain of influenza virus may be resistant to one or more drugs selected from the group consisting of: amantadine, oseltamivir, rimantadine, zanamivir and the like. The drug resistant strain of influenza virus may be a multi-drug resistant strain which may be resistant to a plurality of drugs.

The oligonucleotide sequence may be between 13 and 35 linked nucleotides in length and may comprise at least 70% sequence identity to any of the sequences selected from the group consisting of SEQ ID NO:1 to SEQ ID NO:3. A skilled person will appreciate that a given primer need not hybridize with 100% complementarity in order to effectively prime the synthesis of a complementary nucleic acid strand in an amplification reaction. A primer may hybridize over one or more segments such that intervening or adjacent segments are not involved in the hybridization event, (e.g., for example, a loop structure or a hairpin structure). In particular, the sequence of the oligonucleotide may have 80%, 85%, 90%, 95% or 98% sequence identity to any one of the sequences selected from the group consisting of SEQ ID NO:1 to SEQ ID NO:3.

An extent of variation of 70% to 100%, or any range therewithin, of the sequence identity is possible relative to the specific primer sequences disclosed. Determination of sequence identity is described in the following example: a primer 20 nucleotides in length which is identical to another 20 nucleotides in length primer having two non-identical residues has 18 of 20 identical residues (18/20=0.9 or 90% sequence identity). In another example, a primer 15 nucleotides in length having all residues identical to a 15 nucleotides segment of primer 20 nucleobases in length would have 15/20=0.75 or 75% sequence identity with the 20 nucleotides primer.

Percent homology, sequence identity or complementarity, can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). A skilled person is able to calculate percent sequence identity or percent sequence homology and able to determine, without undue experimentation, the effects of variation of primer sequence identity on the function of the primer in its role in priming synthesis of a complementary strand of nucleic acid for production of an amplification product.

A set of oligonucleotides may comprise a pair of oligonucleotides according to any aspect of the present invention and at least one probe. The probe may be labeled with a fluorescent dye at 5′ and 3′ ends thereof. Examples of the 5′-labeled fluorescent dye may include, but are not limited to, 6-carboxyfluorescein (FAM), hexachloro-6-carboxyfluorescein (HEX), tetrachloro-6-carboxyfluorescein, and Cyanine-5 (Cy5). Examples of the 3′-labeled fluorescent dye may include, but are not limited to, 5-carboxytetramethylrhodamine (TAMRA) and black hole quencher-1,2,3 (BHQ-1,2,3).

The oligonucleotides of the present invention may be used in various nucleic acid amplification techniques known in the art; such as, for example, Polymerase Chain Reaction (PCR), Nucleic Acid Sequence Based Amplification (NASBA), Transcription-Mediated Amplification (TMA), Rolling Circle Amplification (RCA), Strand Displacement Amplification (SDA), thermophilic SDA (tSDA) or Ligation-Mediated Amplification (LMA). The oligonucleotides of the present invention may also be used in a variety of methods known to one of ordinary skill in the art for direct detection of influenza A without amplification through direct hybridization with viral nucleic acids, or to detect DNA or RNA copies of viral nucleic acids, or their complements.

The oligonucleotide according to any aspect of the present invention may be used in a method for the detection of influenza from either a clinical or a culture sample, wherein the clinical samples may include but are not limited to, nasopharyngeal, nasal and throat swabs as well as nasopharyngeal aspirates and washes. The clinical sample may undergo preliminary processing prior to testing to allow more efficient detection of the viral nucleic acid. For example, the sample may be collected and may be added to transport medium to stabilize the virus. Nasopharyngeal, nasal and throat swabs may be added to a transport medium. Nasopharyngeal aspirates and washes may or may not be stabilized by addition of transport medium. Once received at the testing laboratory, the virus may be inactivated and lysed to liberate the viral RNA. The nucleic acid may optionally then be, extracted to remove potential inhibitors or other interfering agents of later assay steps. To perform the methods of the invention, viral nucleic acids may be mixed with components essential for specific detection of influenza A.

The method of detecting the presence of influenza A virus in a biological sample and/or the method of amplifying the anti-viral drug resistant influenza A virus nucleic acid may further comprise a step of analysing the amplified sequence. In particular, the step of analysis may include sequencing. More in particular, the sequencing step may be a method of pyrosequencing using a sequencing primer comprising, consisting essentially of, or consisting of at least one nucleotide sequence of SEQ ID NO:3, fragment(s), derivative(s), mutation(s), and complementary sequence(s) thereof. Use of Pyrosequencing directly on clinical material enabled the measurement of the dynamics of these mixed genotypes without the polluting or diluting effect of viral culture.

The present invention provides at least one kit for the detection of influenza A and/or B virus, the kit comprising at least one oligonucleotide, pair of oligonucleotides or set of oligonucleotides according to any aspect of the present invention. The kit may be used by clinicians to detect human and avian influenza viruses in patients that are afflicted with influenza symptoms. Such kit will include one or more primer and probe sets for the detection of influenza A, drug resistant influenza A, and/or drug resistant influenza A (H1N1).

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

### Samples

Epidemiological and clinical data and daily respiratory swabs were collected as part of the pandemic outbreak plan. Nucleic acids were extracted with an easyMag instrument (Biomerieux). Real time RT-PCR for Influenza A (H1N1) 2009 was carried out with an in-house assay on an Mx3005P instrument (Stratagene, USA). MDCK cells were used for viral culture. Although three methods of sequencing were used, (traditional sequencing with ABI BigDye terminator chemistry, cloning of PCR products with subsequent sequencing and pyrosequencing) it was the use of pyrosequencing directly on RNA extracted from uncultured samples that yielded the results presented below.

—Patient and Virus

The patient was a 28-year-old female American naval officer based in Singapore who had traveled to North California from May 13 through May 18, 2009 and to Hawaii from May 18 through May 25, 2009. She became unwell on May 25, with symptoms of sore throat, myalgia, redness of the right eye and cough with yellowish sputum. She did not notice any fever and had no record of past medical illness. On May 27 (Day 3 of illness), she sought treatment at the naval medical aid station and was referred to the Communicable Disease Center at Tan Tock Seng Hospital, the designated national outbreak management centre, for treatment and isolation.

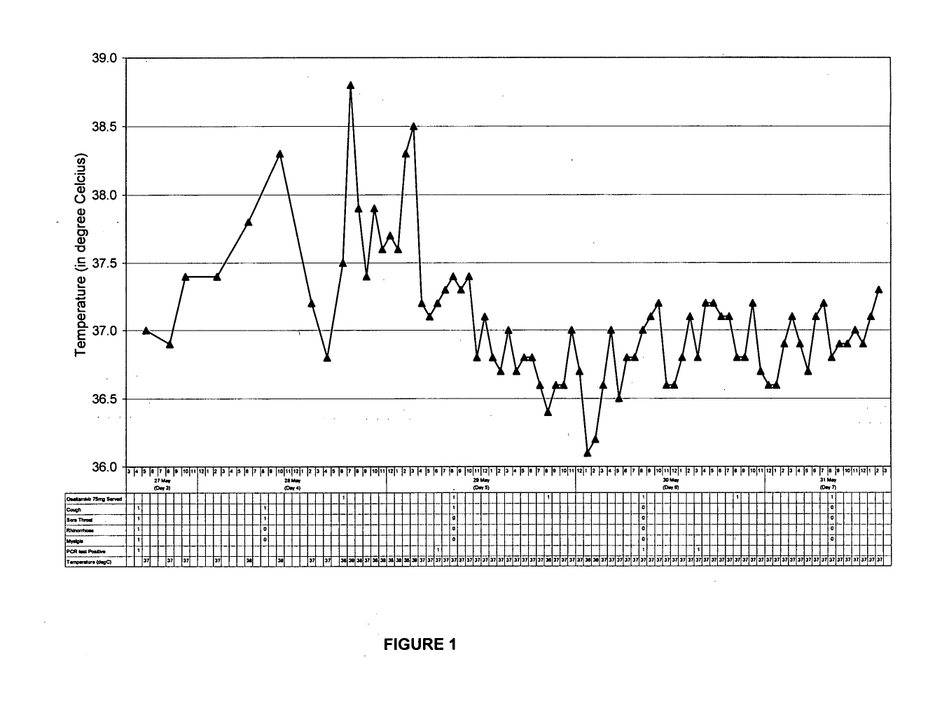

Clinical examination on admission and throughout her hospital stay was unremarkable. A maximum body temperature of 38.8° C. was recorded on day 4 of illness; see FIG. 1. On admission, her leukocyte count was 5300/μl, of which 53.5% were neutrophils and 30.8% lymphocytes. Chest radiography, liver and renal function tests, and C-reactive protein were unremarkable. Two sets of combined nasal and throat swabs taken on the evening of May 27 were reported positive for Influenza A (H1N1) 2009 on May 28th. Oseltamivir treatment was initiated at 6:00 PM later that day. Her fever settled on day 5 of illness and she was discharged from hospital two days later, on May 31st. All 11 close contacts were given oseltamivir prophylaxis and remained well.

The laboratory results are shown in Table 1.

TABLE 1
Results for 6 respiratory samples collected over 4 days.

| | | | | | | | |
|---|---|---|---|---|---|---|---|
| Date | | 27^{th} | 27^{th} | 28^{th} | 29^{th} | 30^{th} | 30^{th} |
| Day of illness | | 3rd | 3rd | 4th | 5th | 6th | 6th |
| Oseltamivir | | nil | nil | 75 mg | 75 mg | 75 mg | |
| RT-PCR | | positive | positive | nd | positive | positive | positive |
| -ct value | | 32 | 33 | — | 24 | 32 | 35 |
| Pyrosequencing | Wild | 100% | 100% | Nd | 100% | 76% | 47% |
| directly on sample | mutant | 0% | 0% | nd | 0% | 23% | 52% |
| Viral Isolation | | nd | nd | + | nd | + | nd |
| Pyrosequence | Wild | — | — | 100% | — | 0% | — |
| of isolated | mutant | — | — | 0% | — | 100% | — |
| virus | | | | | | | |
| Sequencing of | Wild | — | — | 100% | — | — | — |
| clones 192 | mutant | — | — | 0% | — | — | — |
| fragments | | | | | | | |

RT-PCR = reverse transcription PCR for Influenza A (H1N1) 2009;
nd = not done;
ct = cycle threshold. This indicates the relative quantity of virus present. A higher value indicates less virus. Every extra 3.3 cycles represents a decrease in quantity by ten fold.
Mutant = H275Y mutation.

### Creation of Amplicons for Pyrosequencing

RT-PCR was performed with the SuperScript® III One-Step RT-PCR System with Platinum® Taq (Cat. No: 12574-018) in a 50-μl reaction volume containing 5 μl of RNA sample. The biotinylated PCR product was generated with forward primer, (5′-3′)Bio/TGCTTTACTGTAATGACCGAT (SEQ ID NO:1) at a final concentration of 0.25 μM and reverse primer, (5′-3′)GATTCTGGTTGAAAGACACCC (SEQ ID NO:2) at a final concentration of 0.2 μM in a thermal cycler. These primers were designed to obtain high yield of amplicon by single gene amplification. The resulting 217-bp length amplicon accommodates the codon for H275 (CAC) or H275Y (TAC) in the product. An Eppendorf Mastercycler-ep-gradient-S instrument (Hamburg, Germany) was used with the following steps and conditions: reverse transcription at 55° C. for 10 min and initial denaturation at 94° C. for 2.5 min, followed by 40 cycles of: denaturation at 94° C. for 32 s, annealing at 57° C. for 76 sec and extension at 68° C. for 33 sec and a final extension at 68° C. for 5 min. After amplification, the PCR products were analyzed by conventional gel electrophoresis to estimate the yield of the product.

### Pyrosequencing

Pyrosequencing was performed with approximately 200 ng of biotinylated product using the sequencing primer (5′-3′)TAGAATCAGGATAACAGGAGCA (SEQ ID NO:3) according to the manufacturer's guidelines.

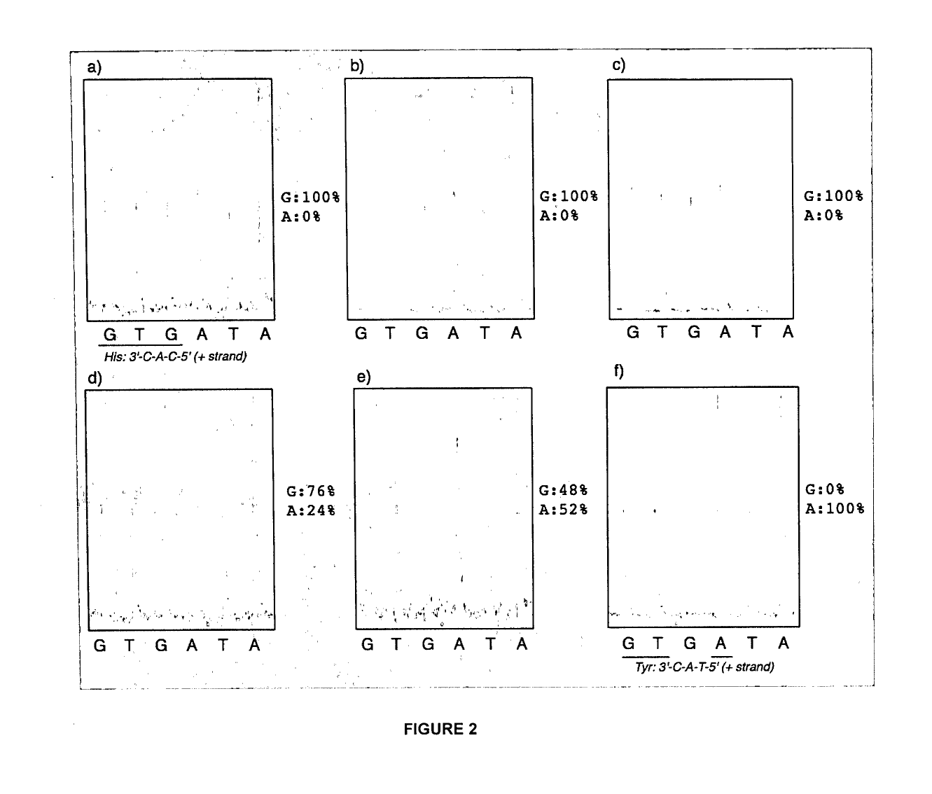

The pyrosequencing sequences are shown in FIG. 2.

Only the wild sequence was detected on samples collected on the day before, the day of and one day after initiation of oseltamivir therapy. The sequence of virus isolated from a sample collected on the day that oseltamivir was started was 100% wild. Mixed wild and mutant sequences were detected by pyrosequencing directly from clinical samples collected 38 and 45 hrs after initiation of therapy; the proportions of mutant virus were 23 and 52% respectively. Virus cultured in vitro from the 38 hr sample appeared to be 100% mutant, even by pyrosequencing.

The patient was discharged well, on the seventh day after the onset of her illness, but the speed with which her mutant emerged and became dominant over the wild virus was considered to be remarkable. The rapid emergence of resistance during treatment in Influenza A H1N1 and the step by step establishment of a predominantly mutant population within 48 hrs of therapy was unexpected and much faster than the 5 days reported with H5N1 (Gupta R. K. et al., 2006). It was also remarkable that the virus isolated from culture of the 38 hr sample was 100% mutant. This was despite the fact that 76% of the virus in the raw sample, prior to culture, was the wild type. This observation that the resistant mutant outgrew the wild type and became the dominant virus in cell culture is testimony to its fitness. Use of pyrosequencing directly on clinical material enabled the measurement of the dynamics of these mixed genotypes without the polluting or diluting effect of viral culture. Although it is an accurate method it still lacks the ability to exclude the presence of minority (<5%) sub-populations (Fakhrai-Rad H et al., 2002). However, the ability of the minority mutant population of 23% in the 38 hr sample to become the only detectable virus in the cultured isolate suggests that if the mutant had been present before therapy with oseltamivir, then it may have been detected it in the culture of the sample collected a few hours prior to therapy with oseltamivir, on day four of illness. However, only wild sequences were detected in virus isolated from this sample and only wild sequences were detected in 192 clones derived directly from the sample prior to culture. This data suggests that the mutation occurred and was selected for after day four.

The ct values in Table 1 show that the amount of virus was at a maximum on day five of illness, approximately a thousand fold greater than on day three and day six. This corresponds to the peak fever which was on days four and five and also to the first day of administration of oseltamivir. The administration of oseltamivir at the time when peak quantities of virus are present is a potent recipe for the selection of resistant mutants.

### REFERENCES

1 WHO Pandemic (H1N1) 2009—update 71. Accessed 30 Oct. 2009. http://www.who.int/csr/disease/swineflu/laboratory23_{—}10_{—}2009/en/print.html
2 Gupta R. K., Nguyen-Van-Tam J. S., de Jong M. D., Hien T. T., Farrar J. Oseltamivir Resistance during Treatment of Influenza A (H5N1) Infection. N Engl J Med 2006; 354: 1423-1424.
3 Influenza A(H1N1) virus resistance to oseltamivir. Summary Table March 2009. Accessed 30 Oct. 2009. http://www.who.int/csr/disease/influenza/h1n1_table/en/index.html
4 Kramarz P, Monnet D, Nicoll A, et al. Use of oseltamivir in 12 European countries between 2002 and 2007—lack of association with the appearance of oseltamivir-resistant influenza A (H1N1) viruses. Eurosurv 2009; 14(5): 1-5.
5 Hurt A C, Ernest J, Deng Y M, et al. Emergence and spread of oseltamivir-resistant A(H1N1) influenza viruses in Oceania, South East Asia and South Africa. Antivir Res 2009; 83:90-3.
6 Fakhrai-Rad H, Pourmand N, Ronaghi M. Pyrosequencing: an accurate detection platform for single nucleotide polymorphisms. Hum Mutat. 2002 May; 19(5):479-85.

1. An isolated oligonucleotide comprising at least one nucleotide sequence selected from the group consisting of: SEQ ID NO:1 to SEQ ID NO:3.

2. The isolated oligonucleotide according to claim 1, wherein the oligonucleotide is capable of binding to and/or being amplified from anti-viral drug resistant Influenza A virus.

3. The isolated oligonucleotide according to claim 2, wherein the anti-viral drug is oseltamivir.

4. The isolated oligonucleotide according to claim 2, wherein the Influenza A virus is of subtype H1N1.

5. A pair of oligonucleotides comprising at least one forward primer and at least one reverse primer, wherein the forward primer comprises SEQ ID NO:1 and the reverse primer comprises SEQ ID NO:2.

6. A pair of oligonucleotides comprising at least one forward primer and at least one reverse primer, wherein the forward primer consists of SEQ ID NO:1 and the reverse primer consists of SEQ ID NO:2.

7. The pair of oligonucleotides according to claim 5, capable of binding to and/or being amplified from anti-viral drug resistant Influenza A virus.

8. The pair of oligonucleotides according to claim 7, wherein the anti-viral drug is oseltamivir.

9. The pair of oligonucleotides according to claim 7, wherein the Influenza A virus is of subtype H1N1.

10. A set of oligonucleotides comprising a pair of oligonucleotides according to claim 5 and at least one probe.

11. An amplicon amplified from Influenza A using at least a pair of oligonucleotides according to claim 5.

12. The amplicon according to claim 11, wherein the Influenza A virus is oseltamivir resistant.

13. A method of detecting the presence of Influenza A in a biological sample, the method comprising the steps of:
(a) providing at least one biological sample;
(b) contacting at least one oligonucleotide according to claim 1, with at least one nucleic acid in the biological sample, and/or with at least one nucleic acid extracted, purified and/or amplified from the biological sample; and
(c) detecting any binding resulting from the contacting in step (b) whereby the Influenza A is present when binding is detected.

14. A method of detecting the presence of Influenza A in a biological sample, the method comprising the steps of:
(a) providing at least one biological sample;
(b) contacting a pair of oligonucleotides according to claim 5, with at least one nucleic acid in the biological sample, and/or with at least one nucleic acid extracted, purified and/or amplified from the biological sample; and
(c) detecting any binding resulting from the contacting in step (b) whereby the Influenza A is present when binding is detected.

15. The method according to claim 14, wherein the virus is anti-viral drug resistant Influenza A virus.

16. The method according to claim 15, wherein the anti-viral drug is oseltamivir.

17. A method of amplifying Influenza A virus nucleic acid, wherein said method comprises carrying out a polymerase chain reaction using a pair of oligonucleotides according to claim 5.

18. A kit for the detection of Influenza A virus, the kit comprising at least one oligonucleotide according to claim 1.

19. A kit for the detection of Influenza A virus, the kit comprising at least one pair of oligonucleotides according to claim 5.
